(19) United States
(12) Plant Patent Application Publication
(10) Pub. No.: US 2002/0152511 P1
(43) Pub. Date: Oct. 17, 2002

(54) MINIATURE VARIETY 'POULflag'

(75) Inventors: L. Pernille Olesen, Fredensborg (DK); Mogens N. Olesen, Fredensborg (DK)

Correspondence Address: Mogens N. Olesen, Poulsen Roser Pacific, Inc., 2550 old Military Road, Central Point, OR, 97502, US

(73) Assignee: Poulsen Roser Aps.

(21) Appl. No.: 09/776,156

(22) Filed: Feb. 2, 2001

(51) Int. Cl. A01H 5/00

(52) U.S. Cl. PLT/119

(57) ABSTRACT

A new miniature rose plant which has abundant, bright orange flowers and attractive foliage. This new and distinct variety has shown to be uniform and stable in the resulting generations from asexual propagation.

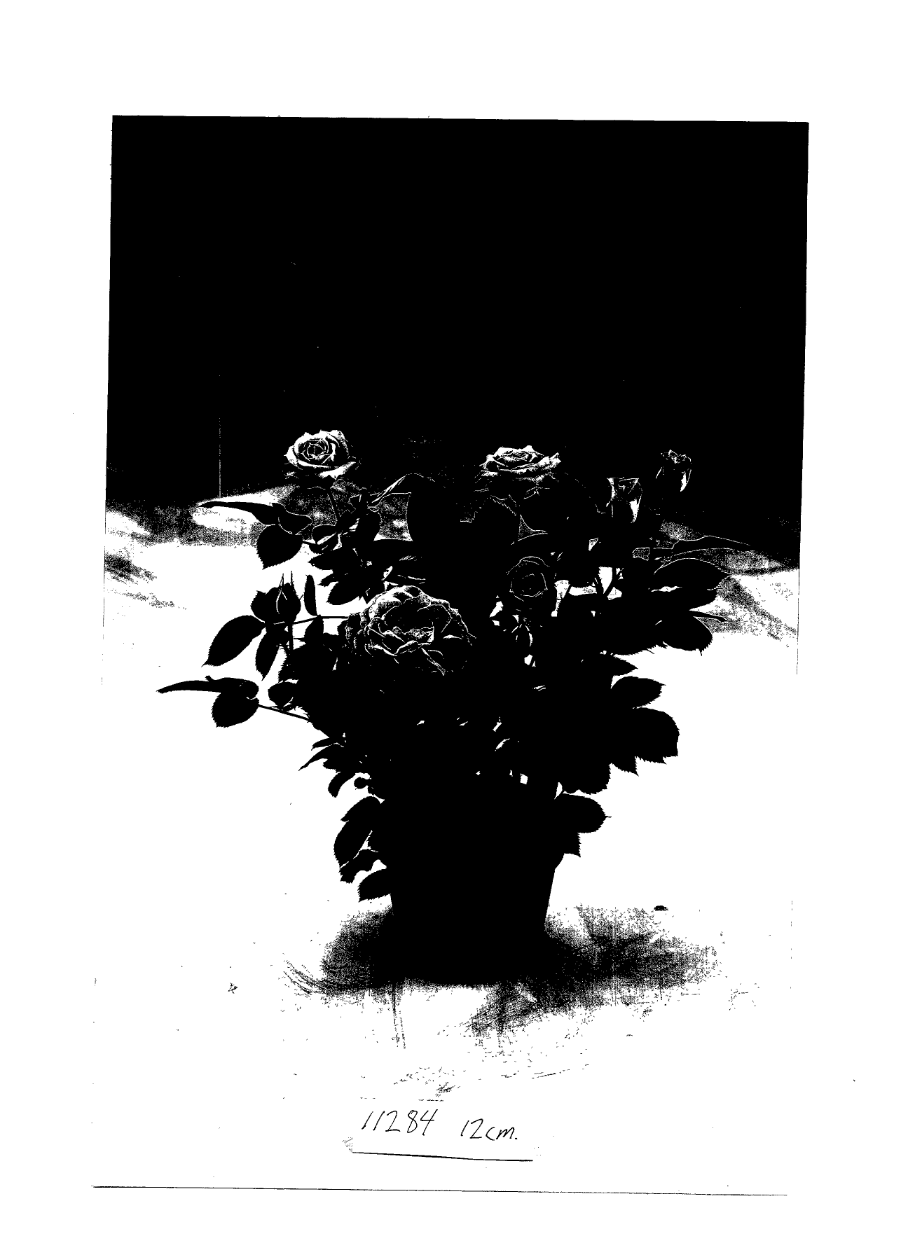

### SUMMARY OF THE INVENTION

The present invention constitutes a new and distinct variety of miniature rose plant which originated from a controlled crossing between ‘POULneto’ and ‘Rumba’, both unpatented varieties. The two parents were crossed during the summer of 1996 and the resulting seeds were planted in a controlled environment in Fredensborg, Denmark. The new variety is named ‘POULflag’.

The new rose may be distinguished from its seed parent, ‘POULneto’, by the following combination of characteristics:

1. ‘POULflag’ has bright orange blooms, whereas the seed parent has soft peach blooms.

2. ‘POULflag’ is a miniature garden rose, whereas ‘POULneto’ is utilized in cut-flower production.

3. ‘POULflag’ exhibits more compact and vigorous growth than the seed parent.

The new variety may be distinguished from its pollen parent, ‘Rumba’ by the following combination of characteristics:

1. ‘POULflag’ has bright orange flowers, whereas ‘Rumba’ has bi-color (yellow/red) flowers.

2. ‘POULflag’ is suitable for pot rose production, while ‘Rumba’ is utilized in cut-flower production.

3. ‘POULflag’ is more compact, even and vigorous in growth when compared ‘Rumba’.

The objective of the hybridization of this rose variety was to create a new and distinct variety for garden use with unique qualities, such as:

1. Uniform and abundant flowers;

2. Vigorous and compact growth when propagated by cuttings;

3. Disease resistance.

This combination of qualities is not present in previously available commercial cultivars of this type and distinguish ‘POULflag’ from all other varieties of which we are aware.

As part of their rose development program, L. Pernille Olesen and Mogens N. Olesen germinated the seeds from the aforementioned hybridization during winter 1996 and conducted evaluations on the resulting seedlings in a controlled environment in Fredensborg, Denmark.

‘POULflag’ was selected in the spring of 1997 by the inventors as a single plant from the progeny of the aforementioned hybridization.

Asexual reproduction of ‘POULflag’ by traditional budding and rooted cuttings was first done by L. Pernille and Mogens N. Olesen in their nursery in Fredensborg, Denmark in summer 1997. This initial and other subsequent asexual propagations conducted in controlled environments have demonstrated that the characteristics of ‘POULflag’ are true to type and are transmitted from one generation to the next.

### BRIEF DESCRIPTION OF THE DRAWING

The accompanying color illustration shows as true as is reasonably possible to obtain in color photographs of this type the variety rose ‘POULflag’. Specifically illustrated in SHEET 1 include:

1. Rose plant exhibiting foliage, leaves, and leaflets.

2. Flower bud, partially opened bud, and open bloom;

3. General plant growth and habit.

### DETAILED DESCRIPTION OF THE VARIETY

The following is a description of ‘POULflag’, as observed in its growth during trials at the testing station Prüfstelle Rethmar under the testing authority Bundessortenamtin in Hannover, Germany and at the inventor's nursery in Fredensborg, Denmark. Color references are made using The Royal Horticultural Society (London, England) Colour Chart, 1995, except where common terms of color are used.

For a comparison, several physical characteristics of the rose variety ‘POULfiry’, a rose variety from the same inventors described and illustrated in U.S. Plant patent application Ser. No. 09/277,237 and dated Mar. 26, 1999 are compared to ‘POULflag’ in Chart 1.

CHART 1

| | ‘POULflag’ | ‘POULfiry’ |
|---|---|---|
| Color of open | Orange Group 28A to | Red Group 40B- |
| flower, inner | Yellow Group 13A | 41B. |
| side, middle | | |
| zone. | | |
| Petalage | 15-25 | 20-25 |
| Upon opening, | Yellow-Orange Group | Yellow-Orange |
| color basal | 13B | Group 22A |
| petal spot, | | |
| outer side | | |

Parents:

Seed parent.— ‘POULneto’.

Pollen parent.— ‘Rumba’.

Classification:

Botanical.—Rosa hybrida.

Commercial.—Miniature.

### FLOWER AND FLOWER BUD

Blooming Habit: Continuous.

Flower Bud:

Bud form.—Pointed to ovate.

Sepals.—Weak appendages.

Receptacle.—Size: Small to medium.

Peduncle.—Surface: Above average abundance of hairs and prickles.

Borne.—Multiple (7-10) buds per stem.

Flower Bloom:

Fragrance.—Slight to fresh perfume.

Size.—Medium. Average flower diameter is 50 mm when open.

Form.—Shape of flower when viewed from the side: Upper part: Flattened convex. Lower part: Flattened convex. Viewed from above: Irregularly rounded.

Petalaqe.—Double. Average range: 15-25 petals under normal conditions.

Color:

Petals.—Inner Side: Middle zone: Orange Group 28A to Yellow Group 13A. Marginal zone: Orange Group 28A to Red Group 40B. Basal petal spot: Yellow-Orange Group 14B. Outer side: Middle zone: Orange Group 24B. Marginal zone: Orange Group 24B to Red Group 40C Basal petal spot: Yellow Group 13B.

General Tonality: Bright orange. On open flower Orange-Red Group 30C. No change in the general tonality.

Petals:

Petal reflex.—Medium to strong.

Undulation of margin.—Medium.

Size.—Small to medium.

Reproductive Organs:

Filaments.—Color: Yellow.

Hips.—Shape: Pitcher-shaped.

### PLANT

Plant Growth: Well-branched, bushy, compact and even in growth. When grown in a 12-15 cm pot, the average height of the plant is 25-30 cm and the average width is 40-60 cm.

Stems:

Thorns.—Incidence: Few. Size: Small to medium. Shape: Concave.

Plant Foliage:

Leaf size.—Medium.

Glossy.—Weak.

Color.—Upper Leaf Surface: Dark green Juvenile foliage: Exhibits anthocyanin on young shoots. Anthocyanin intonation: Color: Bronze to reddish brown.

Plant Leaflets:

Leaflet.—Cross Section: Slight concave. Margin Undulation: Very to medium. Other: Glossy.

Terminal leaflet.—Length of blade: Short to medium. Width of blade: Narrow to medium. Shape: Rounded.

Disease Resistance: Above average resistance to mildew, rust, black spot, and Botrytis under normal growing conditions in Fredensborg, Denmark and Hannover, Germany.

Cold Hardiness: The variety ‘POULflag’ has been found to be cold hardy in Fredensborg, Denmark and Hannover, Germany.

1. A new and distinct variety of rose plant of the miniature rose class, substantially as herein illustrated and described as a distinct and novel rose variety due to its abundant bright orange flowers, disease resistance, and extended period of bloom.
